(19) United States
(12) Patent Application Publication
(10) Pub. No.: US 2019/0060050 A1
(43) Pub. Date: Feb. 28, 2019

(54) CLEAN RELEASE SPLIT CARTON AND METHOD

(71) Applicant: Medtronic Vascular, Inc., Santa Rosa, CA (US)

(72) Inventor: Jeffrey Barnell, Santa Rosa, CA (US)

(21) Appl. No.: 16/058,708

(22) Filed: Aug. 8, 2018

Related U.S. Application Data
(60) Provisional application No. 62/552,259, filed on Aug. 30, 2017.

(51) Int. Cl. A61F 2/00; A61F 2/07; B65D 75/06; B65D 75/58

(52) U.S. Cl. CPC A61F 2/0095; A61F 2/07; A61F 2002/065; B65D 75/5827; B65D 75/06

(57) ABSTRACT

In accordance with one embodiment, a long medical device is sealed within a clean release split carton. The clean release split carton includes a first carton tube coupled to a second carton tube by a closure label. To open the clean release split carton, a tear string is pulled to tear the closure label thus separating the first carton tube from the second carton tube. The individual carton tubes are then removed from the medical device. The clean release split carton avoids the generation of particulates while opening the carton and make removing the medical device easier.

### RELATED APPLICATIONS

This application claims the benefit of U.S. Provisional Application No. 62/552,259, filed on Aug. 30, 2017, entitled “CLEAN RELEASE SPLIT CARTON” of Jeffrey Barnell, which is incorporated herein by reference in its entirety.

### BACKGROUND

### Field

The present application relates to packaging for a medical device and method.

### Description of the Related Art

Physicians and staff involved in aortic stent graft procedures have challenges removing long (58-65″) stent graft delivery systems, e.g. the Valiant™ Thoracic Stent Graft System with the Captivia™ Delivery System and Endurant™ II/Endurant™ IIs bifurcated stent grafts, and other long delivery system products from the shelf carton. Traditionally, an end open carton has been employed where the delivery system is loaded and unloaded from the end.

To remove the delivery system, the operator telescopes the delivery system out of the carton. In doing so, the delivery system and carton effectively become twice as long. If the operator is small in stature or if the operating room (OR)/Stent Lab is cramped, the process becomes cumbersome. There is a risk of damaging the delivery system if it is held improperly or dropped. For disposal, the full-length carton must be bent in half and crushed before recycling.

To address this problem, recent thoracic aortic aneurysm (TAA) and abdominal aortic aneurysm (AAA) programs have moved to a top open carton. In this arrangement, the entire length of the carton opens allowing the operator to lift the pouched delivery system directly out of the carton. To open the carton, the operator must find and tear open two clear tape seals along the long edge. They then begin sliding their fingers between the top and the bottom of the carton, prying them apart as they go. Since most labs do not have a table, this process is typically done with the carton held vertically with one end on the floor and the operator bending over to the floor. In addition to the strained ergonomic position, the carton may stick and then pop open causing the operator to lose control of the device.

### SUMMARY

In accordance with one embodiment, a long medical device is sealed within a clean release split carton. The clean release split carton includes a first carton tube coupled to a second carton tube by a closure label. In one embodiment, the first and second carton tubes have a rectangular cross-section with one end closed although have other shapes in other embodiments.

To open the clean release split carton, a tear string is pulled to tear the closure label thus separating the first carton tube from the second carton tube. One of the carton tubes is removed exposing the end of the medical device which is then pulled from the remaining carton tube. As cutting of the first carton tube from the second carton tube in the operating room is avoided, the clean release split carton is opened easily and without generation of particulates.

Embodiments are best understood by reference to the following detailed description when read in conjunction with the accompanying drawings.

### BRIEF DESCRIPTION OF DRAWINGS

FIG. 1 is a perspective view of a medical device assembly in accordance with one embodiment.

FIG. 2 is a perspective view of a clean release split carton of the medical device assembly of FIG. 1 in a closed state in accordance with one embodiment.

FIG. 3 is a laid-out plan view of the clean release split carton of the medical device assembly of FIG. 1 in accordance with one embodiment.

FIG. 4 is an enlarged laid-out plan view of a region IV of a carton form of the clean release split carton of FIG. 3 in accordance with one embodiment.

FIG. 5 is an enlarged laid-out plan view of the region IV of the clean release split carton of FIG. 3 including the carton form having a rip tape assembly applied thereto in accordance with one embodiment.

FIG. 6 is a cross-sectional view of the clean release split carton of FIG. 2 along the line VI-VI in accordance with one embodiment.

FIG. 7 is a cross-sectional view of the clean release split carton of FIG. 2 along the line VII-VII in accordance with one embodiment.

FIG. 8 is a perspective view of the clean release split carton during opening in accordance with one embodiment.

FIG. 9 is a perspective view of the medical device assembly having the clean release split carton opened in accordance with one embodiment.

FIG. 10 is a perspective view of a medical device assembly including a clean release split carton in accordance with another embodiment.

FIG. 11 is a cross-sectional view of the clean release split carton of FIG. 2 along the line VI-VI in accordance with another embodiment.

Common reference numerals are used throughout the drawings and detailed description to indicate like elements.

### DETAILED DESCRIPTION

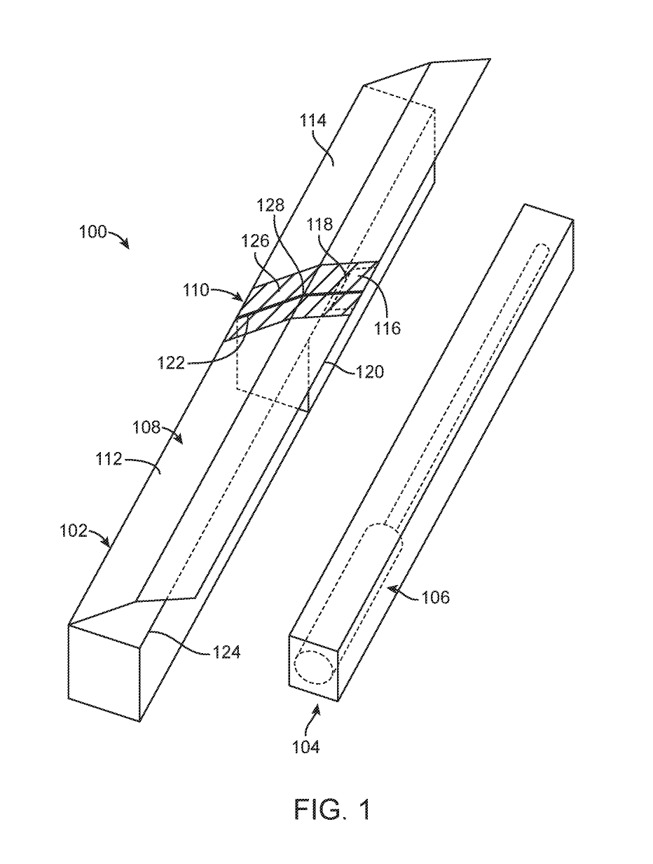

FIG. 1 is a perspective view of a medical device assembly 100 in accordance with one embodiment. Medical device assembly 100 includes a clean release split carton 102, an inner container 104, and a medical device 106. In one embodiment, medical device 106 is an endoluminal delivery system for delivering one or more prosthesis, e.g., stent-grafts for reinforcing weak areas, to a patient. For example, medical device 106 is a long (58-65″) stent graft delivery system, e.g., the Valiant™ Thoracic Stent Graft System with the Captivia™ Delivery System or the Endurant™ II/Endurant™ IIs bifurcated stent graft. Although examples of medical device 106 are provided, in other embodiments, medical device 106 is any one of a number of medical devices. Clean release split carton 102 can be used with medical devices of any size and shape.

Medical device 106 is maintained in a sterile condition within inner container 104 prior to use, e.g., during shipping and storage. Inner container 104 forms a sterile barrier around medical device 106. Inner container 104 can include one or more individual pouches, cartons, or other containers. For example, inner container 104 is a sealed tray such as that disclosed in Barnell, U.S. Patent Application Ser. No. 62/359,049, filed Jul. 7, 2016, entitled “HYBRID SEALED TRAY FOR LONG CATHETER DELIVERY SYSTEMS”, which is herein incorporated by reference in its entirety.

Medical device 106 is contained within inner container 104, which is contained within clean release split carton 102. Clean release split carton 102, sometimes called a box or shelf carton, protects medical device 106 including inner container 104 prior to use. For example, medical device 106 including inner container 104 are contained within clean release split carton 102 during shipping, storage, and generally prior to use. Clean release split carton 102 can include one or more labels to identify the specific medical device 106 contained therein as well as other relevant information.

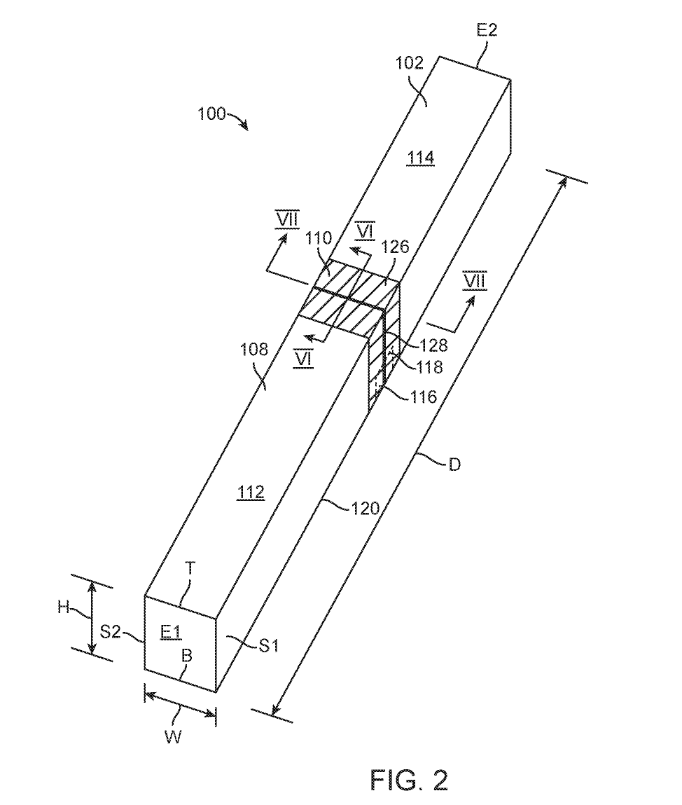

Clean release split carton 102 is illustrated in a semi-closed position in FIG. 1. For example, medical device 106 is contained within inner container 104. Inner container 104 including medical device 106 are placed within clean release split carton 102. After being placed within clean release split carton 102, clean release split carton 102 is sealed as illustrated in FIG. 2. In one embodiment, inner container 104 including medical device 106 are loaded from one of the ends E1, E2 (see FIG. 2) of clean release split carton 102, which is then sealed.

FIG. 2 is a perspective view of clean release split carton 102 of medical device assembly 100 of FIG. 1 in a closed state in accordance with one embodiment. In its closed state, clean release split carton 102 is rectangular having a depth D, a width W, and a height H. In this embodiment, clean release split carton 102 is a long narrow box having depth D much greater than width W and height H. For clarity, a direction parallel to depth D is referred to as a length or depth direction, a direction parallel to width W is referred to as the width direction, and a direction parallel to height H is referred to as the height direction. In accordance with this embodiment, clean release split carton 102 is rectangular and includes a base B, an opposite top T, a first sidewall S1, and opposite second sidewall S2, a first end E1, and an opposite second end E2.

Although a particular shaped clean release split carton 102 is described and illustrated, clean release split carton 102 can take any one of a number of shapes depending upon the particular medical device 106 to be contained therein. Further, clean release split carton 102 does not have a gravitational reference and can be rotated in other embodiments. For example, if rotated 180 degrees, base B would be over top T.

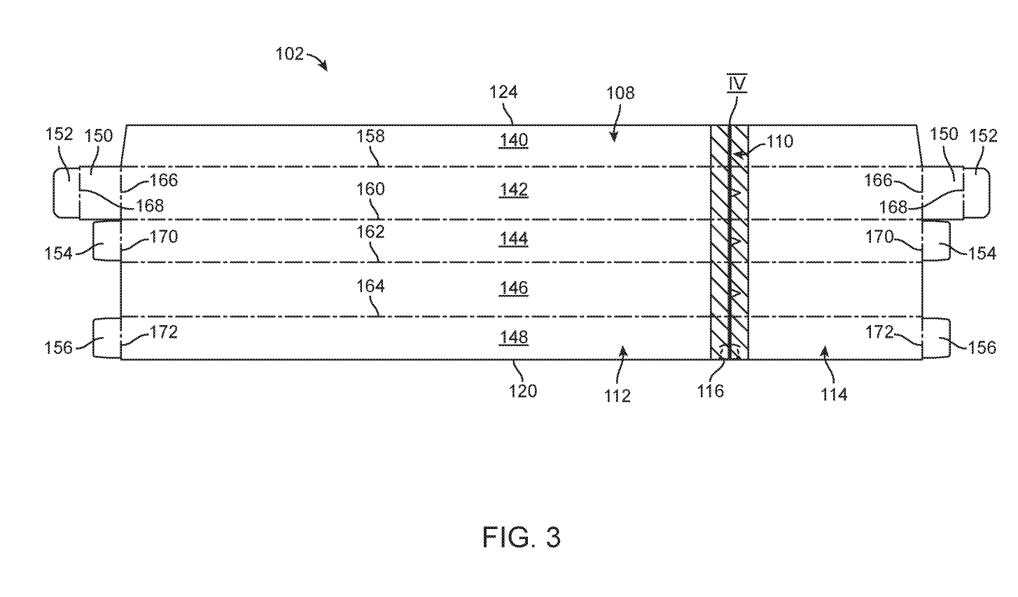

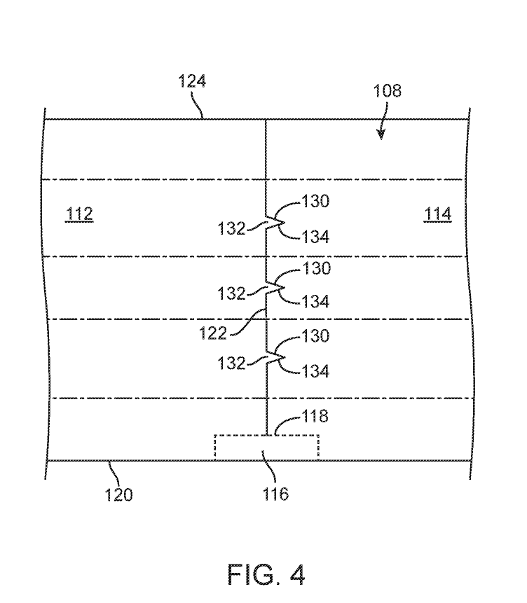

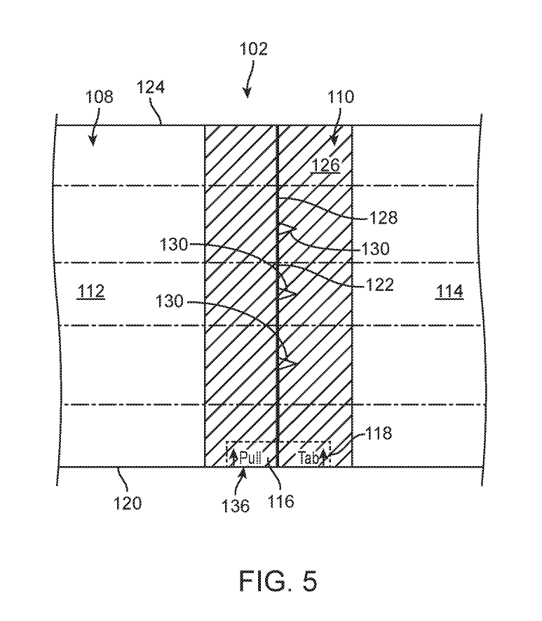

FIG. 3 is a laid-out plan view, also known as a die line view, of clean release split carton 102 of medical device assembly 100 of FIG. 1 in accordance with one embodiment. FIG. 4 is an enlarged laid-out plan view of a region IV of a carton form 108 of clean release split carton 102 of FIG. 3 in accordance with one embodiment. FIG. 5 is an enlarged laid-out plan view of the region IV of clean release split carton 102 of FIG. 3 including carton form 108 having a rip tape assembly 110 applied thereto in accordance with one embodiment. In FIGS. 3-5, a solid line is a cut line, a dash dot line is a fold line, and a dotted line is a perforated cut line (e.g., see 118 in FIG. 4), also called a breakaway 118.

Clean release split carton 102 includes carton form 108 and rip tape assembly 110. Carton form 108 forms the structure of clean release split carton 102 and is fabricated of a rigid sturdy material typically used for packaging such as corrugated cardboard, plastic, box board (e.g., similar to a cereal box), or other packaging material.

Carton form 108 includes a first carton tube 112, a second carton tube 114, and a pull tab 116. In one embodiment, when assembled, first and second carton tubes 112, 114 have a rectangular cross-section with one end closed although have other shapes in other embodiments.

A breakaway 118 is a perforated cut between pull tab 116 and carton tubes 112, 114. In other words, first carton tube 112 and second carton tube 114 are coupled to pull tab 116 by breakaway 118. Breakaway 118 is configured to break upon application of force by the user desiring to remove medical device 106 from clean release split carton 102 as discussed further below to separate pull tab 116 from carton tubes 112, 114.

Pull tab 116 is at a first longitudinal edge 120 of carton form 108. Pull tab 116 is rectangular in this embodiment and is defined by first longitudinal edge 120 and breakaway 118. However, in other embodiments, pull tab 116 has other shapes extending from first longitudinal edge 120.

First carton tube 112 is separated from second carton tube 114 at a release line 122. Release line 122 is a cut in a plane perpendicular to the depth D of clean release split carton 102, i.e., in the height and width direction. Release line 122 extends from pull tab 116 and breakaway 118 to a second longitudinal edge 124 of carton form 108.

First carton tube 112 is completely separated from second carton tube 114 by release line 122 except at pull tab 116. However, rip tape assembly 110 is applied to carton form 108 over release line 122 to hold carton tubes 112, 114 together.

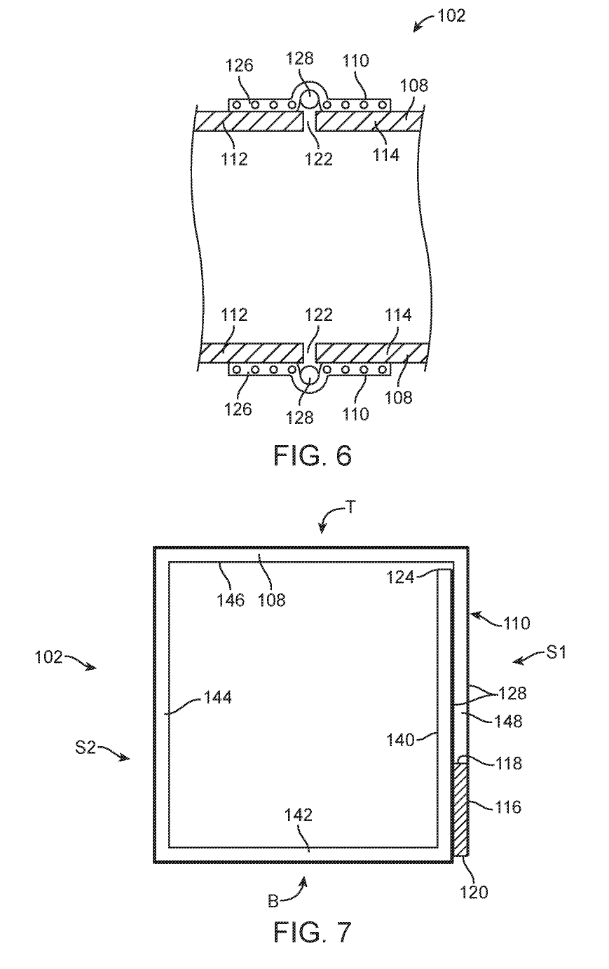

FIG. 6 is a cross-sectional view of clean release split carton 102 of FIG. 2 along the line VI-VI in accordance with one embodiment. FIG. 7 is a cross-sectional view of clean release split carton 102 of FIG. 2 along the line VII-VII in accordance with one embodiment. In FIGS. 6 and 7, inner container 104 and medical device 106 are not illustrated for simplicity

Rip tape assembly 110, sometimes called a custom adhesive backed film, includes a closure label 126 and a tear string 128, sometimes called a pull string 128. Closure label 126 is a single sided adhesive in accordance with this embodiment. Closure label 126 has a specific profile to tear exactly where desired and is die cut to fit exactly around a specific perimeter of clean release split carton 102. Tear string 128 is a flexible but strong cord, sometimes called a high strength thread. Rip tape assembly 110 comes with tear string 128 adhered to closure label 126, i.e., tear string 128 is integrated into closure label 126, in one embodiment. Tear string 128 is embedded in closure label 126 at a specific location. When pulled, tear string 128 shears closure label 126 as discussed further below. Closure label 126 is adhered to carton tubes 112, 114 to hold carton tubes 112, 114 together.

Rip tape assembly 110 may include reinforcing fibers as shown in FIG. 6 that run parallel to tear string 128. Rip tape assembly 110 may also consist of a single film layer, or multiple layers of different composition with or without reinforcement to achieve the desired strength characteristics for a particular application.

Rip tape assembly 110 is applied to carton form 108 such that tear string 128 is located directly over pull tab 116 and release line 122. Tear string 128 is covered by closure label 126. More particularly, tear string 128 is taped above pull tab 116 and release line 122 by closure label 126. Generally, rip tape assembly 110 is applied along release line 122.

Perpendicular to release line 122, i.e., in the depth direction, closure label 126 is very strong. In the direction along the release line, i.e., in the height and width directions, closure label 126 can be easily torn once the tear is initiated.

Closure label 126 tapes first carton tube 112 to second carton tube 114 and holds carton tubes 112, 114 together at release line 122. In accordance with this embodiment, release line 122 is formed with one or more alignment features 130, sometimes called registration details or a keyway, to enhance alignment between first carton tube 112 and second carton tube 114 prior to application of rip tape assembly 110. Alignment features 130 restrict lateral movement of carton tubes 112, 114 prior to taping with rip tape assembly 110.

Each alignment feature 130 includes a protrusion 132 and a corresponding pocket 134 into which protrusion 132 fits. Protrusion 132 and pocket 134 extend in the depth direction from one of cartons tubes 112, 114 to the other carton tubes 112, 114. In accordance with this embodiment, protrusion 132 and pocket 134 are V-shaped, i.e., release line 122 includes one or more V-shapes to define protrusion 132 and pocket 134. However, protrusion 132 and pocket 134 have other shapes in other embodiments. In yet another embodiment, release line 122 is a straight line and carton form 108 is formed without alignment features 130. In applications where the material of carton form 108 is non-fibrous, e.g., a plastic sheet, and therefore does not generate particulates, release line 122 may be significantly perforated along its entire length or short sections may be left uncut or scored. The perforations serve the same function as alignment features 130.

Rip tape assembly 110 extends entirely across carton form 108 from first longitudinal edge 120 to second longitudinal edge 124 of carton form 108. In one embodiment, to form clean release split carton 102, carton form 108 is die cut with first carton tube 112, second carton tube 114, pull tab 116 including breakaway 118 and release line 122 with alignment features 130. Rip tape assembly 110 is applied to carton form 108 immediately after cutting. Carton form 108 including rip tape assembly 110 is glued, folded, and assembled.

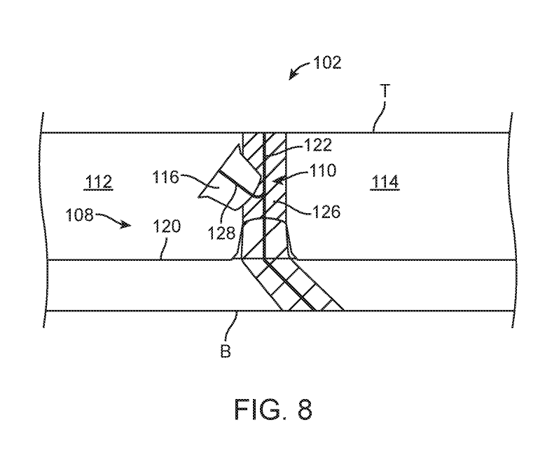

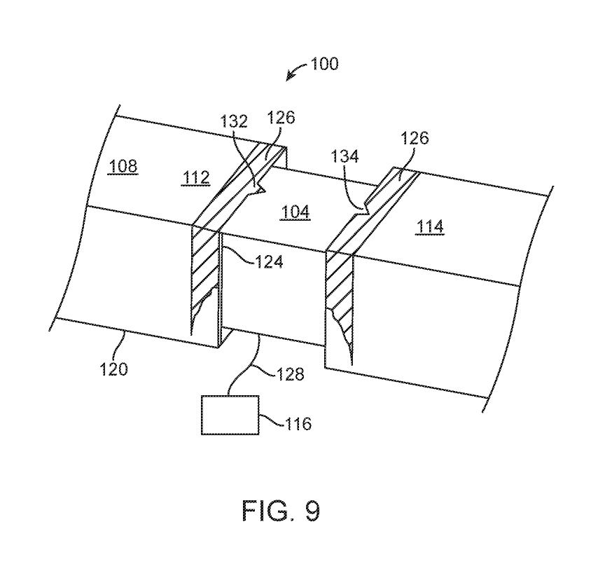

FIG. 8 is a perspective view of clean release split carton 102 during opening in accordance with one embodiment. FIG. 9 is a perspective view of medical device assembly 100 having clean release split carton 102 opened in accordance with one embodiment. Pay particular attention now to FIGS. 2, 8-9 together, to open clean release split carton 102, the user grasps and pulls pull tab 116 to separate pull tab 116. In one embodiment, clean release split carton 102 includes printed graphics 136 (see FIG. 5) that identify pull tab 116 and the direction of pull. Printed graphics 136 are printed on carton form 108 and/or rip tape assembly 110 in various embodiments.

More particularly, upon application of force to pull tab 116, breakaway 118 breaks thus releasing pull tab 116 from carton tubes 112, 114. However, tear string 128 remains attached to pull tab 116.

Pull tab 116 is then pulled by the user to cause tear string 128 to cut through closure label 126 directly above release line 122. Pull tab 116 is pulled until tear string 128 cuts all the way around closure label 126 to second longitudinal edge 124.

In applications using an alignment feature 130, once closure label 126 is severed by tear string 128, first carton tube 112 is completely separated from second carton tube 114. As illustrated in FIG. 9, upon separation of first carton tube 112 from second carton tube 114, first carton tube 112 and second carton tube 114 are easily slid off of inner container 104. In other applications where a perforation is used along release line 122, first carton tube 112 is easily separated along the perforation line from second carton tube 114.

First carton tube 112 is separated from second carton tube 114 without generating any particulates. More particularly, release line 122 is formed during assembly of clean release split carton 102. Accordingly, cutting of carton form 108 and the associated generation of particulates in the operating room is avoided. Closure label 126 is cut cleanly by tear string 128 without generation of particulates. In addition, as only closure label 126 is cut, the force required to open clean release split carton 102 is minimized.

Clean release split carton 102 is separated into two manageable sections, i.e., carton tubes 112, 114. In one embodiment, release line 122 is at or near the center of clean release split carton 102 such that carton tubes 112, 114 are the same length. Accordingly, the need to telescope inner container 104 out of the entire depth D of clean release split carton 102 is eliminated. The empty clean release split carton 102, i.e., carton tubes 112, 114, are short enough to fit directly into a recycle bin without additional crushing or folding in one embodiment.

There is no need to pry a lid and a bottom of clean release split carton 102 apart, nor bend over clean release split carton 102 to reach the far end. Clean release split carton 102 is formed without end seals.

In one embodiment, the movement and steps required to open clean release split carton 102 are minimized thus improving efficiency in the operating room. Clean release split carton 102 is opened easily while clean release split carton 102 and the operator are standing upright. The operator is not required to telescope inner container 104 out of the entire depth D of clean release split carton 102 making clean release split carton 102 more accommodating to petite operators and small labs. The operator maintains better control of clean release split carton 102 while reducing the amount of time needed to remove inner container 104 from clean release split carton 102. This, in turn, provides a labor cost savings.

As discussed above, clean release split carton 102 can have various shapes and features. Various features of carton form 108 as illustrated in FIG. 3 are as follows: an inner first sidewall 140, sometimes called a glue tab 140, a base 142, a second sidewall 144, a cover 146, an outer first sidewall 148, outer end walls 150, outer end wall closure tabs 152, second sidewall closure tabs 154, and first sidewall closure tabs 156. Inner first sidewall 140 is coupled to base 142 by a fold 158. Base 142 is coupled to second sidewall 144 by a fold 160. Second sidewall 144 is coupled to cover 146 by a fold 162. Cover 146 is coupled to outer first sidewall 148 by a fold 164. Note that folds 158, 160, 162, 164 extend in the depth direction in the constructed and closed clean release split carton 102.

Outer end walls 150 are coupled to base 142 by folds 166. Outer end wall closure tabs 152 are coupled to outer end walls 150 by folds 168. Note that folds 166, 168 extend in the width direction in the constructed and closed clean release split carton 102.

Second sidewall closure tabs 154 are coupled to second sidewall 144 by folds 170. First sidewall closure tabs 156 are coupled to the outer first sidewall 148 by folds 172. Note that folds 170, 172 extend in the height direction in the constructed and closed clean release split carton 102.

To assembly clean release split carton 102, glue is applied to inner first sidewall 140 except at the location where pull tab 116 will abut. Clean release split carton 102 is then folded such that outer first sidewall 148 lands on inner first sidewall 140 having adhesive applied thereto. As the location where pull tab 116 contacts inner first sidewall 140 has an absence of adhesive, pull tab 116 is not adhered to inner first sidewall allowing the user to grasp and remove (pull) pull tab 116.

In one embodiment, clean release split carton 102 is less expensive than a standard carton. For example, clean release split carton 102 contains 50% less material than a standard top open carton and is assembled in 15 seconds versus 3-5 minutes for a standard top open carton. Clean release split carton 102 is delivered preassembled in one embodiment.

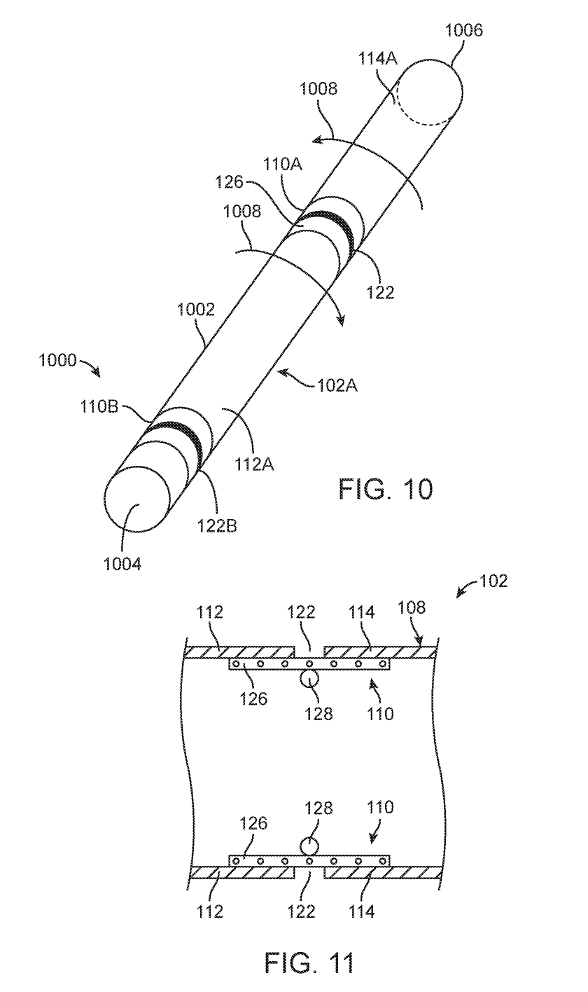

FIG. 10 is a perspective view of a medical device assembly 1000 including a clean release clean release split carton 102A in accordance with another embodiment. Medical device assembly 1000 is of FIG. 10 is similar to medical device assembly 100 of FIGS. 1-2 and only the significant differences are discussed below.

In accordance with this embodiment, clean release split carton 102A is cylindrical in shape having a cylindrical outer surface 1002 and circular ends 1004, 1006. A rip tape assembly 110A is applied over release line 122.

In accordance with this embodiment, rip tape assembly 110A does not include a tear string, i.e., includes closure label 126 only. To open clean release split carton 102A, the operator simply twists a first carton tube 112A from a second carton tube 114A as indicated by the arrows 1008 to break rip tape assembly 110A, e.g., closure label 126. This separates first carton tube 112A from second carton tube 114A.

In FIG. 10, in accordance with one embodiment, clean release split carton 102A include a second rip tape assembly 110B applied over a second release line 122B. In this embodiment, clean release split carton 102A includes multiple rip tape assemblies 110A, 110B for separate compartments. For example, a documentation compartment is located at end 1004 and a medical device compartment is located at end 1006. Rip tape assembly 110B allows access to the documentation compartment, whereas rip tape assembly 110A allows access to the medical device compartment. In one embodiment, clean release split carton 102 of FIG. 2 includes multiple rip tape assemblies for multiple compartments in a similar manner.

FIG. 11 is a cross-sectional view of clean release split carton 102 of FIG. 2 along the line VI-VI in accordance with another embodiment. In accordance with this embodiment, rip tape assembly 110 is applied inside of carton form 108. For example, closure label 126 is a double sided adhesive that holds carton tubes 112, 114 together on one side and hold tear string 128 on the other side. In accordance with this embodiment, tear string 128 is pulled to cut closure label 126 and tear string 128 is pull out through release line 122.

This disclosure provides exemplary embodiments. The scope is not limited by these exemplary embodiments. Numerous variations, whether explicitly provided for by the specification or implied by the specification or not, such as variations in structure, dimension, type of material and manufacturing process may be implemented by one of skill in the art in view of this disclosure.

What is claimed is:

1. A medical device assembly comprising:
a clean release split carton comprising:
a first carton tube;
a second carton tube separated from the first carton tube by a release line;
a rip tape assembly over the release line holding the first carton tube and the second carton tube together.

2. The medical device assembly of claim 1 further comprising a carton form comprising the first carton tube and the second carton tube.

3. The medical device assembly of claim 2 wherein the carton form further comprises a pull tab.

4. The medical device assembly of claim 3 wherein the pull tab is coupled to the first carton tube and the second carton tube by a breakaway.

5. The medical device assembly of claim 4 wherein the breakaway is a perforated cut configured to break upon application of force to separate the pull tab from the first carton tube and the second carton tube.

6. The medical device assembly of claim 5 further comprising printed graphics that identify the pull tab and the direction of pull.

7. The medical device assembly of claim 3 where the rip tape assembly is located over the pull tab.

8. The medical device assembly of claim 1 wherein the rip tape assembly comprises a closure label adhered to the first carton tube and the second carton tube.

9. The medical device assembly of claim 8 where the rip tape assembly further comprises a tear string over the release line.

10. The medical device assembly of claim 1 further comprising one or more alignment features for alignment of the first carton tube to the second carton tube.

11. The medical device assembly of claim 10 wherein the release line comprises the one or more alignment features.

12. The medical device assembly of claim 1 wherein the clean release split carton comprises a carton form having the rip tape assembly inside.

13. The medical device assembly of claim 1 further comprising a medical device contained within the clean release split carton.

14. The medical device assembly of claim 13 further comprising an inner container within the clean release split carton, the medical device being within the inner container.

15. A medical device assembly comprising:
a carton form comprising:
a first longitudinal edge;
a second longitudinal edge;
a pull tab at the first longitudinal edge; and
a release line extending from the pull tab to the second longitudinal edge; and
a rip tape assembly extending from the first longitudinal edge to the second longitudinal edge over the pull tab and the release line.

16. The medical device assembly of claim 15 wherein the rip tape assembly comprises:
a closure label; and
a tear string configured to tear the closure label.

17. The medical device assembly of claim 15 wherein the carton form further comprises:
a first carton tube;
a second carton tube separated from the first carton tube by the release line;
a breakaway coupling the pull tab to the first carton tube and the second carton tube.

18. A method comprising:
forming a clean release split carton comprising coupling a first carton tube to a second carton tube with a rip tape assembly;
sealing a medical device within the clean release split carton; and
opening the clean release split carton to remove the medical device comprising tearing the rip tape assembly to separate the first carton tube from the second carton tube.

19. The method of claim 18 wherein the tearing comprises separating a pull tab from the first carton tube and the second carton tube, the rip tape assembly being coupled to the pull tab.

20. The method of claim 19 wherein the rip tape assembly comprises a tear string and a closure label coupled to the pull tab, the method further comprising pulling the pull tab to cause the tear string to tear the closure label.
